(19) United States
(12) Patent Application Publication
(10) Pub. No.: US 2025/0303110 A1
(43) Pub. Date: Oct. 2, 2025

(54) Catheter Clearing System and Methods

(71) Applicant: C. R. Bard, Inc., Franklin Lakes, NJ (US)

(72) Inventors: Robin Raj Mariya David, Srivilliputtur (IN); Vinod Kumar Subramanian, Madurai (IN); Kumara Gurubaran Shanmuga Sundaram, Madurai (IN)

(21) Appl. No.: 18/618,839

(22) Filed: Mar. 27, 2024

(51) Int. Cl. A61M 25/00

(52) U.S. Cl. CPC A61M 25/0017; A61M 25/0023

(57) ABSTRACT

Catheter lumen clearing systems include a catheter and a lumen clearing system. The catheter includes a catheter tube defining a lumen. The lumen clearing system comprises a shaft extending longitudinally through the catheter lumen, a handle disposed at a proximal end of the shaft, and a head disposed at a distal end of the shaft. The outer surface of the head is configured to engage the inner surface of the catheter lumen as the head is urged proximally through the catheter lumen. This engagement allows the head to effectively clear a deposit from the catheter lumen, ensuring unobstructed flow and optimal performance of the catheter.

### BACKGROUND

Previous approaches for clearing deposits from catheters have involved various methods and devices. One common approach is the use of mechanical clearing devices, such as brushes or wires, which are inserted into the catheter lumen and manually manipulated to remove the deposits. While these mechanical devices can be effective in some cases, they often require significant force and manual dexterity to operate, which can be challenging for healthcare professionals and may cause discomfort or injury to the patient. Further, they often are introduced in a distal direction risking displacement of the deposits towards the body cavity.

Another approach involves the use of chemical agents or solutions to dissolve or break down the deposits within the catheter lumen. These methods typically involve the introduction of a liquid or gel into the catheter, which is allowed to sit for a period of time to facilitate the dissolution of the deposits. While these clearing methods can be effective for certain types of deposits, they may not be suitable for all types of catheters or deposits, and there is a risk of damage to the catheter or surrounding tissues if the active ingredient is not properly selected or used.

Additionally, some previous approaches have utilized a combination of mechanical and chemical clearing methods. These systems typically involve the use of a mechanical clearing device in conjunction with a chemical agent to enhance the clearing process. While these combined methods may provide improved clearing capabilities, they can still be time-consuming and may require multiple steps or components to achieve the desired result.

Disclosed herein are catheter clearing systems and associated methods directed to address the foregoing.

### SUMMARY

In some aspects, the techniques described herein relate to a catheter clearing system including, a catheter having a catheter tube defining a catheter lumen, and a lumen clearing system including, a shaft extending longitudinally through the catheter lumen, a handle disposed at a proximal end of the shaft, and a head disposed at a distal end of the shaft, an outer surface of the head configured engage an inner surface of the catheter lumen as the head is urged proximally through the catheter lumen to clear a deposit.

In some aspects, the techniques described herein relate to a catheter clearing system, wherein the catheter is one of an intermittent catheter, Foley catheter, balloon catheter, indwelling catheter, peritoneal catheter, or pleural drainage catheter.

In some aspects, the techniques described herein relate to a catheter clearing system, wherein the catheter is configured to drain urine from a bladder of a patient.

In some aspects, the techniques described herein relate to a catheter clearing system, wherein the catheter further includes a funnel disposed at a proximal end of the catheter tube in fluid communication with the catheter lumen.

In some aspects, the techniques described herein relate to a catheter clearing system, wherein the catheter further includes one or more eyelets disposed in a side wall of the catheter tube and provides fluid communication with the catheter lumen.

In some aspects, the techniques described herein relate to a catheter clearing system, further including a coupling configured to secure one or both of the handle and a proximal portion of the shaft to an outer surface of the catheter, adjacent a proximal end.

In some aspects, the techniques described herein relate to a catheter clearing system, wherein the coupling includes an adhesive tape having a tear line and configured to adhere the proximal portion of the shaft to the outer surface of the catheter.

In some aspects, the techniques described herein relate to a catheter clearing system, wherein the coupling includes a clip molded into an outer surface of the catheter and configured to releasably secure the proximal portion of the shaft using an interference-fit engagement or a snap-fit engagement.

In some aspects, the techniques described herein relate to a catheter clearing system, further including a grip slidably engaged with an outer surface of the catheter tube.

In some aspects, the techniques described herein relate to a catheter clearing system, wherein a proximal surface of the head defines a toroidally-shaped concave surface extending about a central longitudinal axis of the head to define an acute angle to a radial outer edge of the head.

In some aspects, the techniques described herein relate to a catheter clearing system, further including a retaining ring disposed on an inner surface of the catheter lumen and extending annularly, the retaining ring configured to abut against a portion of the head to releasably secure the head at a distal end of the catheter lumen.

In some aspects, the techniques described herein relate to a catheter clearing system, wherein the head further includes one or more skives extending longitudinally along an outer surface of the head.

In some aspects, the techniques described herein relate to a catheter clearing system, further including a loading jig configured to urge the head and a distal portion of the shaft distally through the catheter lumen and position the head at a distal most point of the catheter lumen.

In some aspects, the techniques described herein relate to a method of clearing a lumen of a catheter including, grasping a handle of a lumen clearing system, the handle coupled to a proximal end of a shaft, the shaft including a head disposed at a distal end, urging the handle proximally to urge the head proximally, an outer surface of the head engaging an inner surface of the catheter lumen and removing a deposit therefrom, and urging the head to a proximal end of the catheter lumen to remove the deposit and restore patency to the catheter lumen.

In some aspects, the techniques described herein relate to a method, wherein the catheter includes a catheter tube defining the catheter lumen and a funnel disposed at a proximal end of the catheter tube in fluid communication with the catheter lumen.

In some aspects, the techniques described herein relate to a method, further including draining a fluid through an eyelet disposed in a side wall of the catheter tube to enter the catheter lumen.

In some aspects, the techniques described herein relate to a method, wherein the catheter is a one of an intermittent catheter, Foley catheter, balloon catheter, indwelling catheter, peritoneal catheter, or pleural drainage catheter.

In some aspects, the techniques described herein relate to a method, further including draining urine from a bladder of a patient.

In some aspects, the techniques described herein relate to a method, further including detaching one or both of the handle and a proximal portion of the shaft from a coupling, prior to urging the handle proximally, the coupling releasably securing the one or both of the handle and the proximal portion of the shaft to an outer surface of the catheter.

In some aspects, the techniques described herein relate to a method, wherein the coupling is an adhesive tape and detaching one or both of the handle and a proximal portion of the shaft from the coupling further includes breaching a tear line extending through the adhesive tape.

In some aspects, the techniques described herein relate to a method, wherein a proximal surface of the head defines a toroidally-shaped concave surface extending about a central longitudinal axis of the head to define an acute angle to a radial outer edge.

In some aspects, the techniques described herein relate to a method, wherein urging the head proximally further includes disengaging the head from a retaining ring disposed at a distal end of the catheter lumen, the retaining ring abutting against the head to releasably retain the head at a distal end of the catheter lumen.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of a catheter system generally including a catheter, a catheter clearing system and a loading jig, in accordance with embodiments disclosed herein.

FIG. 2 shows a perspective view of the catheter clearing system of FIG. 1, in accordance with embodiments disclosed herein.

FIG. 3 shows close up detail of the proximal end of the catheter system of FIG. 1, in accordance with embodiments disclosed herein.

FIG. 4 shows a longitudinal cross-section view of a distal end of the catheter system of FIG. 1, in accordance with embodiments disclosed herein.

FIG. 5 shows a perspective view of a distal end of the catheter system of FIG. 1 with the catheter shown in wire frame for ease of illustration, in accordance with embodiments disclosed herein.

FIG. 6A shows close up detail of a distal end of a catheter clearing system, in accordance with embodiments disclosed herein.

FIG. 6B shows a longitudinal cross-section view of a distal end of a catheter clearing system, in accordance with embodiments disclosed herein.

FIG. 7 shows a lateral cross-section view of a distal end of the catheter and catheter clearing system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter or system disclosed herein includes a portion of the catheter or system intended to be near or relatively nearer to a clinician when the catheter or system is used on a patient. Likewise, a “proximal length” of, for example, the catheter or system includes a length of the catheter or system intended to be near or relatively nearer to the clinician when the catheter or system is used on the patient. A “proximal end” of, for example, the catheter or system includes an end of the catheter or system intended to be near or relatively nearer to the clinician when the catheter or system is used on the patient. The proximal portion, the proximal end portion, or the proximal length of the catheter or system can include the proximal end of the catheter or system; however, the proximal portion, the proximal end portion, or the proximal length of the catheter or system need not include the proximal end of the catheter or system. That is, unless context suggests otherwise, the proximal portion, the proximal end portion, or the proximal length of the catheter or system is not necessarily a terminal portion or terminal length of the catheter or system.

With respect to “distal,” a “distal portion” or a “distal end portion” of, for example, a catheter or system disclosed herein includes a portion of the catheter or system intended to be near or relatively nearer to a patient when the catheter or system is used on a patient. Likewise, a “distal length” of, for example, the catheter or system includes a length of the catheter or system intended to be near or relatively nearer to the patient when the catheter or system is used on the patient. A “distal end” of, for example, the catheter or system includes an end of the catheter or system intended to be near or relatively nearer to the patient when the catheter or system is used on the patient. The distal portion, the distal end portion, or the distal length of the catheter or system can include the distal end of the catheter or system; however, the distal portion, the distal end portion, or the distal length of the catheter or system need not include the distal end of the catheter or system. That is, unless context suggests otherwise, the distal portion, the distal end portion, or the distal length of the catheter or system is not necessarily a terminal portion or terminal length of the catheter or system.

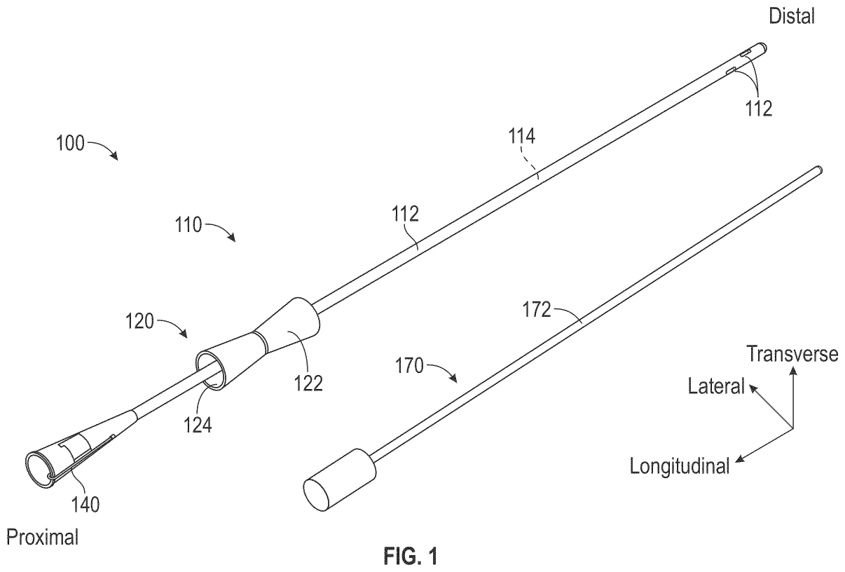

To assist in the description of embodiments described herein, as shown in FIG. 1, a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

FIG. 1 shows a perspective view of a catheter system 100 generally including a catheter 110 and a lumen clearing system 140. The catheter system 100 can be configured to access a cavity within a body of a patient and drain a fluid therefrom. For example, a distal end of the catheter can be placed into a bladder of a patient to drain urine therefrom. However, these examples are not intended to be limiting and it will be appreciated that other catheters, body cavities, and drainage fluids are also contemplated to fall within the scope of the present invention. Exemplary catheters include intermittent catheters, Foley catheters, balloon catheters, indwelling catheters, peritoneal catheters, pleural drainage catheters, or the like.

The catheter 110, or portions thereof can be formed of a flexible material such as a plastic, polymer, rubber, elastomer, silicone rubber, or the like. The catheter 110 can be flexible enough to extend through tortuous pathways and access the cavity of the body. Further, the catheter 110 can display sufficient columnar strength to allow an axial force to urge the catheter 110 through the tortuous pathways and access the cavity of the body without kinking or collapsing.

In an embodiment, the catheter 110 includes a catheter tube 112 extending along a longitudinal axis and defining a catheter lumen 114. A distal end of the catheter tube 112 includes one or more eyelets 116 extending through a side wall of the catheter tube 112 and providing fluid communication with the catheter lumen 114. It will be appreciated that various, numbers, configurations, and positions of eyelets are also contemplated to fall within the scope of the present invention. A proximal end of the catheter tube 112 includes a funnel 118. Optionally the proximal end of the funnel 118 includes a coupling that allows a fluid collection container, collection bag, drainage tube, or combinations thereof to be coupled thereto and collect a fluid from the catheter lumen 114.

In an embodiment, the catheter further includes a grip 120. The grip 120 includes a grip body 122 defining a grip lumen 124. A portion of the catheter 110 extends through the grip lumen 124 and is slidably engaged therewith. A first, or proximal end, of the grip lumen 124 defines a flared, or funnel-shape. Further, a second, or distal end, of the grip lumen 124 also defines a flared, or funnel-shape. Advantageously, the grip 120 facilitates holding and manipulating the catheter 110 during placement without having to directly contact the catheter tube 112, mitigating infection.

In an embodiment, the catheter system 100 further includes a lumen clearing system 140 configured to facilitate the clearing of biofilms, mucus, or similar deposits disposed within the catheter lumen 114 which might affect the patency or flow rate of drainage fluids therefrom, or might fully occlude the catheter lumen 114 altogether. Advantageously, the lumen clearing system 140 facilitates the mechanical clearing of a catheter lumen 114 without requiring additional medications to breakdown and remove the deposits therefrom. This mitigates the excess use of catheter clearing medications and any associated side-effects or contraindications, such as damage to medical devices or surrounding tissues. Advantageously, the catheter system 100 facilitates the mechanical clearing of a catheter lumen 114 prior to or simultaneously with the use of catheter clearing medications to increase the efficacy of these medications, and/or requiring a lower dose of the catheter clearing medications, mitigating any associated side-effects, contraindications, or damage to surrounding medical devices or tissues.

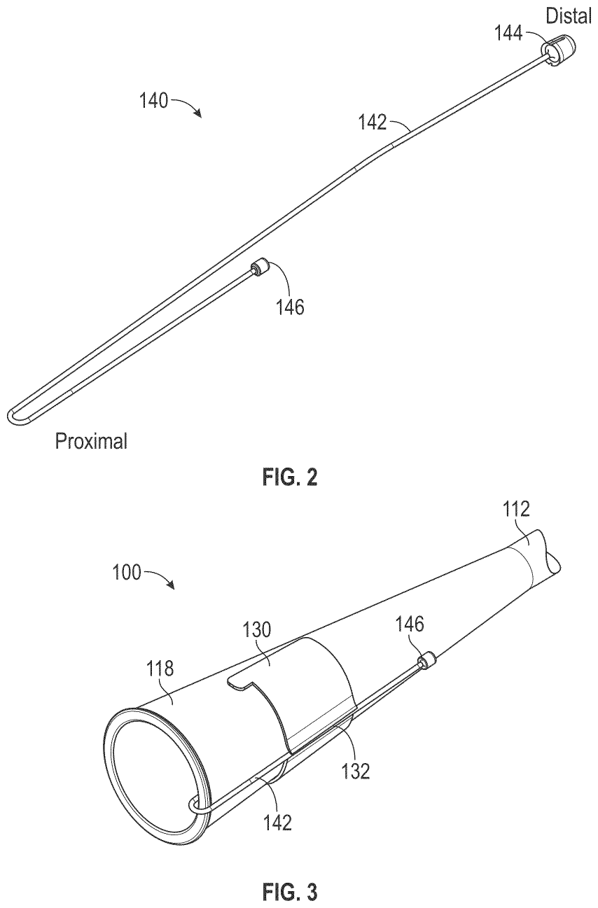

As shown in FIG. 2, the lumen clearing system 140 generally includes a shaft 142 extending longitudinally between a head 144, disposed at a distal end, and a handle 146, disposed at a proximal end. The head 144 can be disposed at a distal end of the catheter lumen 114 with the shaft 142 extending proximally therefrom, through the catheter lumen 114, to a proximal end of the funnel 118. As shown in FIG. 1, a proximal portion of the shaft 142 including the handle 146, can be folded back on itself to extend along an outer surface of the catheter 110. To note the configuration of the lumen clearing system 140 as shown in FIG. 2 includes the proximal portion folded back on itself such that the handle 146 is disposed distally as if the lumen clearing system 140 were disposed within the catheter 110. The catheter 110 is not shown in FIG. 2 for case of illustration.

The lumen clearing system 140, or portions thereof, can be formed of a plastic, polymer, polypropylene, nylon polyamide, metal, alloy, composite, or the like. In an embodiment, the shaft 142 can be formed of a single strand of material, or a plurality of strands of woven or twisted together, e.g., to form a string, wire, or rope. In an embodiment, the lumen clearing system 140 can be formed of the same material, or can include two or more different materials. For example, the shaft 142 can be formed of two or more different materials twisted together to form a string. In an embodiment, the shaft 142 can be flexible, elastically deformable, or malleable to fit within the catheter 110 and allow the catheter 110 to maintain sufficient flexibility in order to extend through tortuous pathways to access the body cavity of the patient.

In an embodiment, the shaft 142 can display increased tensile strength to allow the shaft 142 to be a thin as possible while still allowing the head 144 to be pulled through the catheter lumen 114 without the shaft 142 rupturing. In an embodiment, the shaft 142 can display increased columnar strength to allow the head 144 to be urged distally through the catheter lumen 114 to a distal end thereof, without buckling or collapsing.

As shown in FIG. 2, the handle 146 includes a plug formed integrally with a proximal end of the shaft 142 and configured to facilitate grasping the proximal end of the shaft 142. However, it will be appreciated that the handle 146 can also include other shapes or structures, including a T-shaped handle, finger loop, or similar structure configured to facilitate grasping a proximal end of the shaft 142. The handle 146 can be formed of the same material, or of a different material, from that of the shaft 142.

As shown in FIG. 3, in an embodiment, one or both of the handle 146 and a proximal portion of the shaft 142 extends along an outer surface of the catheter 110 and can be releasably coupled therewith. As shown in FIG. 3, one or both of the handle 146 and a proximal portion of the shaft 142 are releasably coupled in place using a coupling 130, such as an adhesive tape. However, it will be appreciated that the coupling 130 can also include adhesives, latches, clips, snap-clips, interference fit engagement, press-fit engagements, snap-fit engagements, combinations thereof, or the like. In an embodiment, the coupling can be formed integrally with the outside of the catheter 110, such as a snap-fit clip configured to releasably retain a proximal portion of the shaft 142. In an embodiment, the coupling can be formed as a separate structure and coupled with the catheter 110.

In an embodiment, the coupling 130 includes an adhesive tape that is disposed over one or both of the handle 146 and a proximal portion of the shaft 142 and secures the handle 146 and/or portion of the shaft 142 to the outer surface of the funnel 118. In an embodiment, the coupling 130 further includes a tear line 132, such as a perforation, laser cut line, or similar line of weakness extending therethrough and aligned with the handle 146 and/or portion of the shaft 142 disposed therebelow. Advantageously, the tear line 132 facilitates separation of the adhesive tape therealong, releasing the handle 146 and/or portion of the shaft 142 disposed therebelow when a user grasps the handle 146 and urges the handle 146 away from the outer surface of the catheter 110.

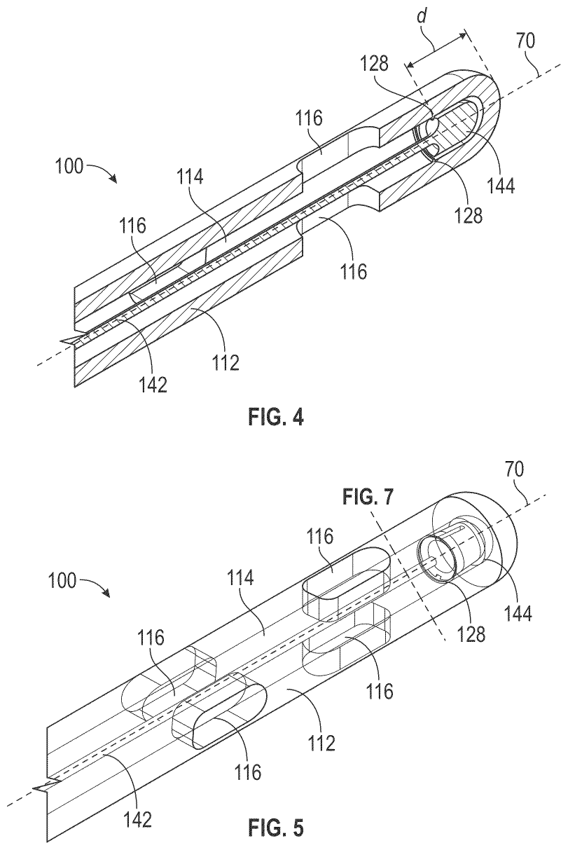

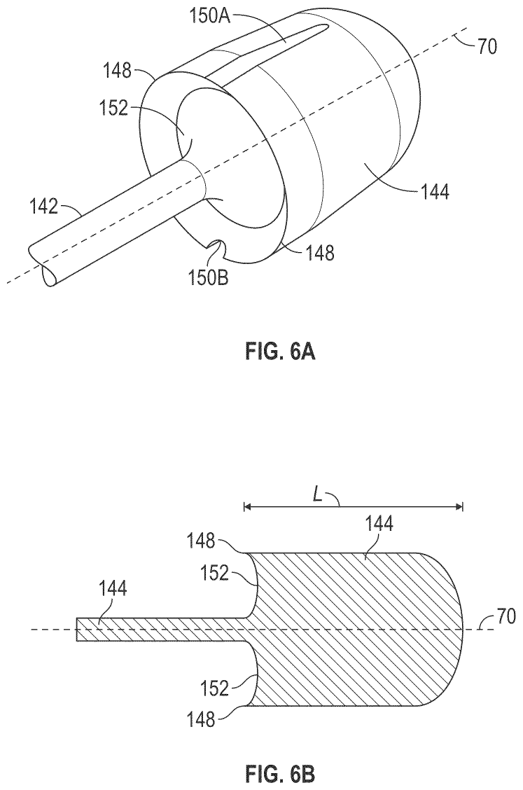

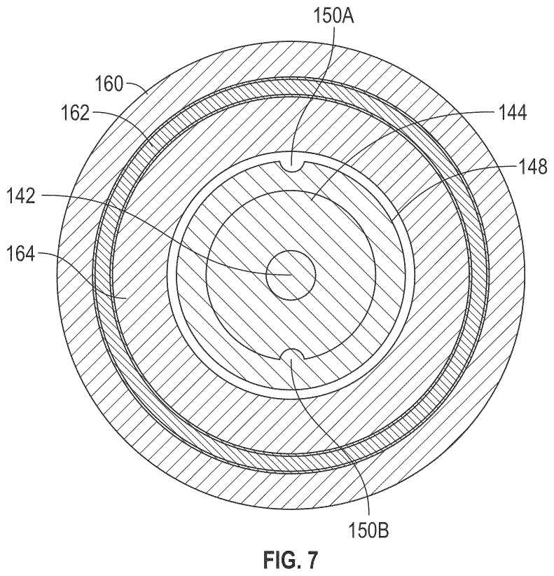

FIGS. 4-7 show further details of the distal end of the lumen clearing system 140. FIG. 4 shows a longitudinal cross-section view of the head 144 disposed at a distal end of the catheter lumen 114. FIG. 5 shows a perspective view of the head 144 disposed at a distal end of the catheter lumen 114 with the catheter shown in wire frame for case of illustration. FIG. 6A shows a perspective view of the head 144. FIG. 6B shows a longitudinal cross-section view of the head 144. FIG. 7 shows a lateral cross-section view of the head 144 disposed at a distal end of the catheter lumen 114.

The head 144 defines a substantially cylindrical shape, or slightly conical shape, having a circular lateral cross-sectional shape to match the circular lateral cross-sectional shape of the catheter lumen 114. An outer diameter of the head 144 is equal to, or slightly larger than an inner diameter of the catheter lumen 114. As such, the head 144 fits tightly within the catheter lumen 114 and is slidably engaged therewith. In an embodiment, the radial outer surface of the head 144 contacts the inner wall of the catheter lumen 114 along at least a portion of the circumference of the head 144. In an embodiment, the outer surface of the head 144 contacts the catheter lumen 114 along an entire circumference of the head 144.

In an embodiment, as shown in FIGS. 4-5, the inner surface of the catheter lumen 114 includes a retaining ring 128 extending annularly about a central longitudinal axis 70 and disposed proximally of the head 144 when the head 144 is disposed at the distal-most end of the catheter lumen 114. Worded differently, the retaining ring 128 is disposed at a distance (d) (FIG. 4) from the distal end of the catheter lumen 114, where the distance (d) is slightly larger than a length (L) (FIG. 6B) between a distal tip of the head 144 and a proximal edge 148 of the head 144. Advantageously, the retaining ring 128 is configured to abut against the proximal radial outer edge 148 in a snap-fit engagement to releasably retain the head 144 at the distal end of the catheter lumen 114 during transport, storage, and/or placement of the catheter 110. When the lumen clearing system 140 is activated, a user can apply an axial force to the shaft 142 to urge the head 144 proximally and urge the head past the retaining ring 128. Advantageously, the retaining ring 128 retains the head distally of the distal-most eyelet 116 to prevent the head 144 from occluding the eyelet 116 and inhibiting proximal fluid flow through the catheter lumen 114.

In an embodiment, a distal tip of the head 144 defines a semi-spherical shape, configured to match the semi-spherical shape of the inner surface of the distal tip of the catheter lumen 114. In an embodiment, a distal tip of the catheter 110 includes a distal eyelet (not shown) extending therethrough and aligned with a longitudinal axis of the catheter 110.

In an embodiment, as shown in FIGS. 6A-6B, a proximal surface 152 of the head 144 includes a toroidal concave shape extending about a central longitudinal axis 70. As such a radial outer edge 148 of the proximal surface defines an acute angle when viewed in longitudinal cross-section. Advantageously, the radial outer edge 148 of the proximal surface provides a scraping edge configured to facilitate removing deposits from the inner surface of the catheter lumen 114 as the head 144 is urged proximally.

In an embodiment, as shown in FIG. 6A, an outer surface of the head 144 includes one or more skives, for example a top skive 150A and a bottom skive 150B. The skives 150A, 150B allow and radial outer edge 148 to flex radially inward as the head 144 is urged proximally past the retaining ring 128, while still allowing a majority of the radially outer edge 148 to maintain a circular shape and maintain contact with the catheter lumen inner surface without buckling or kinking.

In an embodiment, the skives 150A, 150B also serve as a pressure release feature. When the head 144 is being urged proximally to clear deposits within the catheter lumen 114, there is a significant increase in drag as the amount of deposits being urged proximally ahead of the head 144 increases. To prevent the drag force exceeding a tensile strength of the shaft 142, the skives 150A, 150B allow a small amount of the deposits to be urged past the head 144 and allow the head 144 to pass through the catheter lumen 114 without the shaft 142 rupturing and blocking the catheter lumen 114 entirely. Similarly, when resetting the lumen clearing system 140 by urging the head 144 distally through the catheter lumen 114, when the head 144 reaches the distal end of the catheter lumen 114, any deposits trapped between the head 144 and the distal end of the catheter lumen 114 are allowed to pass through the skives 150A, 150B and can be cleared on a subsequent activation of the lumen clearing system 140. This allows the head 144 to be seated properly, distally of the retaining ring 128.

FIG. 7 shows a lateral cross-section view of the head 144 disposed in a distal end of the catheter lumen 114. In an embodiment, the outer diameter of the head 144 is slightly smaller than an inner diameter of the catheter lumen 114. In an embodiment, the wall of the catheter tube 112 includes an outer layer of silicone rubber 160, and middle layer of resin 162 and an inner layer of silicone rubber 164. The resin layer 162 allows the outer layer of silicone rubber 160 to move relative to the inner layer of silicone rubber 164 and allows the catheter tube 112 to flex perpendicular to the longitudinal axis to negotiate tortuous pathways and access the cavity of the body.

In an embodiment, a wall thickness of the catheter tube 112 can be reduced and an inner diameter of the catheter lumen 114 can be increased by between 1% and 8% relative to a conventional catheter of similar French size or usage. This increased catheter lumen 114 size allows the catheter 110 to accommodate the cross-sectional size of the shaft 142 within the catheter lumen 114, without reducing the patency or flow rate through the catheter lumen 114. In an embodiment, the inner diameter of the catheter lumen 114 can be increased by 4% relative to a conventional catheter of similar French size or usage type. Exemplary usage types include pediatric, female, male, etc.

In an exemplary method of use, a catheter system 100 is provided as described herein. The user places the catheter 110 to access a cavity of a body of the patient to drain a fluid therefrom. For example, a distal tip of the catheter tube 112 is urged through the urethra to access a bladder. The eyelets 116 provide fluid communication between the bladder and the catheter lumen 114 to drain urine proximally through the catheter lumen 114 and through the funnel 118. During use, deposits can form on the inside of the catheter lumen 114, for example biofilms, mucus layers, or similar deposits. These deposits can reduce patency and fluid flow through the catheter lumen 114, or can occlude fluid flow altogether.

Advantageously, a user can actuate the lumen clearing system 140 to clear the deposits from the catheter lumen 114 and restore patency to the catheter lumen 114. To activate the lumen clearing system 140, a user can grasp the handle 146 at a proximal end of the catheter system 100 and optionally release the handle from the coupling 130. The user can then urge the handle 146 proximally to urge a portion of the shaft 142 and the head 144 through the catheter lumen 114. The user applies sufficient proximal force to urge the head 144 proximally past the retaining ring 128. As the head 144 is urged proximally, the radial outer edge 148 of the proximal surface 152 of the head 144 scrapes any deposits from the inner surface of the catheter lumen 114 and urges the deposits proximally, and out of the catheter lumen 114. Once the lumen clearing system 140 is removed from the catheter lumen 114, patency has been restored to the catheter tube 112 and the user can continue to use the catheter 110.

In an embodiment, once the lumen clearing system 140 has been removed from the catheter 110, the shaft 142 provides sufficient columnar strength to allow the head 144 to be urged back into the catheter lumen 114 to a distal end thereof and allow the lumen clearing system 140 to be used a second time. In an embodiment, the lumen clearing system 140 is a single use system that is disposed of once removed from the catheter 110 and the catheter 110 can be disposed of once removed from the body of the patient. In an embodiment, both the lumen clearing system 140 and the catheter 110 can be cleaned and sterilized after use and reassembled as shown in FIG. 1. In an embodiment, as shown in FIG. 1 the system 100 further includes a loading jig 170, such as a stylet or pressurized air gun, or the like. The loading jig includes a loading shaft 172 configured to be inserted in a distal direction into the catheter lumen 114 and urge the head 144 and a portion of the shaft 142 distally through the catheter lumen 114 to place the head 144 at a distal end of the catheter lumen 114. In an embodiment, the loading jig 170 is used to load the lumen clearing system 140 into the catheter 110 after the catheter 110 is formed and before transport to the end user. In an embodiment, the system 100 is provided with the loading jig 170 to allow an end user to reload the lumen clearing system 140 back into the catheter 110 after use and once it has been cleaned and sterilized.

Advantageously, the lumen clearing system 140 allows a user to restore patency to the catheter 110 without having to remove and re-catheterize the cavity, reducing medical waste and associated costs, and reducing the additional pain and discomfort of re-catheterizing the body cavity.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter clearing system, comprising:
a catheter having a catheter tube defining a catheter lumen; and
a lumen clearing system comprising:
a shaft extending longitudinally through the catheter lumen;
a handle disposed at a proximal end of the shaft; and
a head disposed at a distal end of the shaft, an outer surface of the head configured engage an inner surface of the catheter lumen as the head is urged proximally through the catheter lumen to clear a deposit.

2. The catheter clearing system according to claim 1, wherein the catheter is one of an intermittent catheter, Foley catheter, balloon catheter, indwelling catheter, peritoneal catheter, or pleural drainage catheter.

3. The catheter clearing system according to claim 1, wherein the catheter is configured to drain urine from a bladder of a patient.

4. The catheter clearing system according to claim 1, wherein the catheter further includes a funnel disposed at a proximal end of the catheter tube in fluid communication with the catheter lumen.

5. The catheter clearing system according to claim 1, wherein the catheter further includes one or more eyelets disposed in a side wall of the catheter tube and provides fluid communication with the catheter lumen.

6. The catheter clearing system according to claim 1, further including a coupling configured to secure one or both of the handle and a proximal portion of the shaft to an outer surface of the catheter, adjacent a proximal end.

7. The catheter clearing system according to claim 6, wherein the coupling includes an adhesive tape having a tear line and configured to adhere the proximal portion of the shaft to the outer surface of the catheter.

8. The catheter clearing system according to claim 6, wherein the coupling includes a clip molded into an outer surface of the catheter and configured to releasably secure the proximal portion of the shaft using an interference-fit engagement or a snap-fit engagement.

9. The catheter clearing system according to claim 1, further including a grip slidably engaged with an outer surface of the catheter tube.

10. The catheter clearing system according to claim 1, wherein a proximal surface of the head defines a toroidally-shaped concave surface extending about a central longitudinal axis of the head to define an acute angle to a radial outer edge of the head.

11. The catheter clearing system according to claim 1, further including a retaining ring disposed on an inner surface of the catheter lumen and extending annularly, the retaining ring configured to abut against a portion of the head to releasably secure the head at a distal end of the catheter lumen.

12. The catheter clearing system according to claim 1, wherein the head further includes one or more skives extending longitudinally along an outer surface of the head.

13. The catheter clearing system according to claim 1, further including a loading jig configured to urge the head and a distal portion of the shaft distally through the catheter lumen and position the head at a distal most point of the catheter lumen.

14. A method of clearing a lumen of a catheter, comprising:
grasping a handle of a lumen clearing system, the handle coupled to a proximal end of a shaft, the shaft including a head disposed at a distal end;
urging the handle proximally to urge the head proximally, an outer surface of the head engaging an inner surface of the catheter lumen and removing a deposit therefrom; and
urging the head to a proximal end of the catheter lumen to remove the deposit and restore patency to the catheter lumen.

15. The method according to claim 14, wherein the catheter includes a catheter tube defining the catheter lumen and a funnel disposed at a proximal end of the catheter tube in fluid communication with the catheter lumen.

16. The method according to claim 15, further including draining a fluid through an eyelet disposed in a side wall of the catheter tube to enter the catheter lumen.

17. The method according to claim 14, wherein the catheter is a one of an intermittent catheter, Foley catheter, balloon catheter, indwelling catheter, peritoneal catheter, or pleural drainage catheter.

18. The method according to claim 14, further including draining urine from a bladder of a patient.

19. The method according to claim 14, further including detaching one or both of the handle and a proximal portion of the shaft from a coupling, prior to urging the handle proximally, the coupling releasably securing the one or both of the handle and the proximal portion of the shaft to an outer surface of the catheter.

20. The method according to claim 19, wherein the coupling is an adhesive tape and detaching one or both of the handle and a proximal portion of the shaft from the coupling further includes breaching a tear line extending through the adhesive tape.

21. The method according to claim 14, wherein a proximal surface of the head defines a toroidally-shaped concave surface extending about a central longitudinal axis of the head to define an acute angle to a radial outer edge.

22. The method according to claim 14, wherein urging the head proximally further includes disengaging the head from a retaining ring disposed at a distal end of the catheter lumen, the retaining ring abutting against the head to releasably retain the head at a distal end of the catheter lumen.
